(19) United States
(12) Patent Application Publication
(10) Pub. No.: US 2012/0045516 A1
(43) Pub. Date: Feb. 23, 2012

(54) COMPOSITION FOR PHOTODYNAMIC THERAPY COMPRISING A MACROMOLECULAR CAPSULE

(75) Inventors: Kimoon Kim, Gyeongsangbuk-Do (KR); Kangkyun Baek, Daegu-city (KR); Gyeongwon Yun, Gyeongsangbuk-Do (KR); Youngkook Kim, Seoul (KR); Raghunandan Hota, Gyeongsangbuk-Do (KR); Kyeng Min Park, Gyeongsangbuk-Do (KR); Hyuntae Jung, Gyeongsangbuk-Do (KR)

(73) Assignee: POSTECH ACADEMY-INDUSTRY FOUNDATION, Pohang-si, Kyungsangbuk-do (KR)

(21) Appl. No.: 13/264,427

(22) PCT Filed: Oct. 20, 2009
(86) PCT No.: PCT/KR2009/006032

§ 371 Date: Oct. 14, 2011

(30) Foreign Application Priority Data
Apr. 15, 2009 (KR) 10-2009-0032959

(51) Int. Cl. A61K 9/51; A61K 31/506; A61P 35/00; B32B 27/00; A61K 31/337; A61K 31/704; B82Y 5/00

(52) U.S. Cl. 424/497; 514/449; 514/252.18; 514/34; 428/402; 977/788; 977/906

(57) ABSTRACT

A composition for photodynamic therapy including a polymer capsule having a diameter of about 10 nm to about 2000 nm synthesized by copolymerization of a flat aromatic compound represented by Formula 1 (see the specification) and an organic compound represented by Formula 2 (see the specification).

### TECHNICAL FIELD

The present invention relates to a composition for photodynamic therapy, and more particularly, to a composition for photodynamic therapy containing a polymer capsule formed by copolymerization of planar ring molecules.

The present invention is a result of the research undertaken as part of the Mid-career Researcher Program/Take-off Research Support Program organized by the Korean Ministry of Education, Science, and Technology.

[Project ID No.: 20090051704, Title of Project: Supramolecular Chemistry Toward “Smart” Materials]

### BACKGROUND ART

Photodynamic therapy that uses photosensitizer, which is to kill cancer cells with toxic reactive oxygen species generated by exposure of the photosensitizer to a specific wavelength of light, may solve the problems of side effects or aftereffects with existing standard cancer therapies, including surgery, radiotheraphy, or medication, and thus may prolong life and improve the quality of life of patients without need for a complicated operation (J. Porphyrins Phthalocyanines, 2001, 5, 105).

The use of photosensitizers for photodynamic therapy in cancer treatment was officially approved in 1993, and has been prevalent in several developed countries, including the U.S.A., European countries, and Japan, for treatment of some cancer species and early cancers, with newly developed photosensitizers currently under clinical tests for approval. (Nat. Rev. Cancer 2003, 3, 380). Currently, in Korea, only twelve hospitals have begun to apply photodynamic therapy on cancer patients. However, with the trend of an increasing number of chemistry-majored personnel avoiding getting involved in research, there is an immediate need for expanding investment to research expenses and personnel in this field.

Photofrin, the commonest FDA-approved, commercial photosensitizer, is known as a mixture of porphyrin derivatives. Although currently being used in the treatment of different types of cancers, photofrin has not been understood fully in terms of its composition and may exhibit toxicity in response to light of 630 nm, thus being inappropriate to treat cancer at locations deep in the body. Photofrin may remain in the body for 2 to 3 weeks after completion of the treatment, and in particular, may nonspecifically accumulate in the skin or eyes, thereby causing a photosensitive reaction in the skin, which may inconvenience the patient in having to live in dark conditions after the treatment (J. Natl. Cancer Inst. 1998, 90, 889).

To address these drawbacks, many researchers began to research more into new, efficient photosensitizers (Cancer Res. 2006, 66, 7225; Proc. Natl. Acad. Sci., USA 2007, 104, 8989; J. Am. Chem. Soc. 2007, 129, 7220; J. Am. Chem. Soc. 2008, 130, 4236). However, no FDA-approved photosensitizer has been found yet, except for photofrin, and existing photosensitizers have drawbacks of low selectivity to cancer cells and remaining for too long in the body or too early elimination from the body. If intravenously injected into the body, the photosensitizer may nonspecifically accumulate in a small amount in the skin and eyes, thereby causing side effects. To solve these problems, research for linking polymer with a photosensitizer has been conducted. However, there is still a problem of using excess polymer with no pharmaceutical efficacy.

The inventors of the present invention have found that a polymer capsule having a size of about 10 nm to about 2000 nm may be formed by copolymerization of a flat aromatic compound of Formula 1 below and a compound of Formula 2 without a template or an auxiliary agent (Angew. Chem. Int. Ed. 2007; KR 721431):

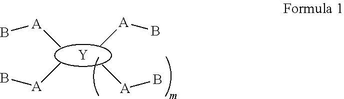

In Formula 1 above,

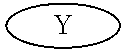

is a C₅-C₅₄ aryl group, or a C₅-C₅₄ heteroaryl group with at least one heteroatom selected from among N, O, and S;

A is independently selected from the group consisting of —(CH₂)ₙ—, —(CH₂)ₙ—S—(CH₂)ₙ—, —(CH₂)ₙ—O—(CH₂)ₙ—, —(CH₂)ₙ—N—(CH₂)ₙ—, —O—, —O—(CH₂)ₙ—, —O—(CH₂)ₙ—C═C—, —O—(CH₂)ₙ—O—, —O—CO—(CH₂)ₙ—, —O—CO—O—(CH₂)ₙ—, —O—(CH₂)ₙ—NH—, —O—(CH₂)ₙ—S—(CH₂)ₙ—COO—, and —(CH₂)ₙ—S—(CH₂)ₙ—NH—, wherein n is an integer from 0 to 30;

B is —CH═CH₂ or —C≡CH;

m is an integer from 0 to 20,

(HS)ⱼ—Z—(SH)ₖ Formula 2

In Formula 2, Z is an unsubstituted or substituted C₁-C₂₀ alkylene, in which O, S, or N may be inserted into the middle of a C—C bond, and the substituent may be selected from the group consisting of —SCH₂CH₂CH₂CH₂CH₂CH₂SH, —SCH₂CH₂OCH₂CH₂OCH₂CH₂SH, —SCH₂CH₂(OH)CH₂(OH)CH₂SH, —CH₂CH₂C(CH₂OOCCH₂CH₂SH)₃, and —C(CH₂OOCCH₂CH₂SH)₃; and

j and k are each independently an integer from 1 to 3.

### DETAILED DESCRIPTION OF THE INVENTIVE CONCEPT

### Technical Problem

The inventors of the present invention have completed the prevent invention as a result of research into new photodynamic therapeutic agents capable of overcoming the above-described drawbacks of existing photodynamic therapeutic agents.

The present invention provides a photodynamic therapeutic agent with improved therapeutic effects and far less side effects as compared to existing photodynamic therapeutic agents and which may remain in the body for a sufficient period of time and may lack unnecessary polymer moieties.

### Technical Solution

According to an aspect of the inventive concept, there is provided a composition for photodynamic therapy including a polymer capsule having a diameter of about 10 nm to about 2000 nm synthesized by copolymerization of a compound represented by Formula 1 below and a compound represented by Formula 2 below:

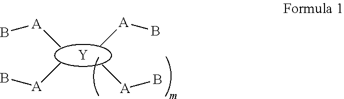

wherein, in Formula 1,

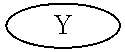

is a C₅-C₅₄ aryl group, or a C₅-C₅₄ heteroaryl group with at least one heteroatom selected from among N, O, and S; A is independently selected from the group consisting of —(CH₂)ₙ—, —(CH₂)ₙ—S—(CH₂)ₙ—, —(CH₂)ₙ—O—(CH₂)ₙ—, —(CH₂)ₙ—N—(CH₂)ₙ—, —O—, —O—(CH₂)ₙ—, —O—(CH₂)ₙ—C═C—, —O—(CH₂)ₙ—O—, —O—CO—(CH₂)ₙ—, —O—CO—O—(CH₂)ₙ—, —O—(CH₂)ₙ—NH—, —O—(CH₂)ₙ—S—(CH₂)ₙ—COO—, and —(CH₂)ₙ—S—(CH₂)ₙ—NH—, wherein n is an integer from 0 to 30; B is —CH═CH₂ or —C≡CH; and m is an integer from 0 to 20,

(HS)ⱼ—Z—(SH)ₖ Formula 2

wherein, in Formula 2, Z is an unsubstituted or substituted C₁-C₂₀ alkylene, in which O, S, or N may be inserted into the middle of a C—C bond, and the substituent is selected from the group consisting of —SCH₂CH₂CH₂CH₂CH₂CH₂SH, —SCH₂CH₂OCH₂CH₂OCH₂CH₂SH, —SCH₂CH₂(OH)CH₂(OH)CH₂SH, —CH₂CH₂C(CH₂OOCCH₂CH₂SH)₃, and —C(CH₂OOCCH₂CH₂SH)₃; and j and k are each independently an integer from 1 to 3.

Hereinafter, embodiments of the present invention will now be described in greater detail.

As a result of research for developing new photodynamic therapeutic agents, the inventors of the present invention found that a polymer capsule disclosed in KR 721431 has a highly effective photodynamic therapeutic activity.

According to an aspect of the present invention, there is provided a composition for photodynamic therapy including a polymer capsule having a diameter of about 10 nm to about 2000 nm synthesized by copolymerization of a compound of Formula 1 below and an aliphatic compound having at least two thiol groups:

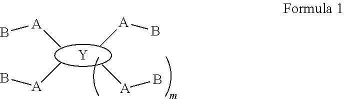

In Formula 1,

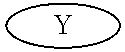

is a C₅-C₅₄ aryl group, or a C₅-C₅₄ heteroaryl group with at least one heteroatom selected from among N, O, and S;

A is independently selected from the group consisting of —(CH₂)ₙ—, —(CH₂)ₙ—S—(CH₂)ₙ—, —(CH₂)ₙ—O—(CH₂)ₙ—, —(CH₂)ₙ—N—(CH₂)ₙ—, —O—, —O—(CH₂)ₙ—, —O—(CH₂)ₙ—C═C—, —O—(CH₂)ₙ—O—, —O—CO—O—(CH₂)ₙ—, —O—(CH₂)ₙ—NH—, —O—(CH₂)ₙ—S—(CH₂)ₙ—COO—, and —(CH₂)ₙ—S—(CH₂)ₙ—NH—, wherein n is an integer from 0 to 30;

B is —CH═CH₂ or —C≡CH; and

m is an integer from 0 to 20,

(HS)ⱼ—Z—(SH)ₖ Formula 2

In Formula 2, Z is an unsubstituted or substituted C₁-C₂₀ alkylene, in which O, S, or N may be inserted into the middle of a C—C bond, and the substituent may be selected from the group consisting of —SCH₂CH₂CH₂CH₂CH₂CH₂SH, —SCH₂CH₂OCH₂CH₂OCH₂CH₂SH, —SCH₂CH₂(OH)CH₂(OH)CH₂SH, —CH₂CH₂C(CH₂OOCCH₂CH₂SH)₃, and —C(CH₂OOCCH₂CH₂SH)₃; and

j and k are each independently an integer from 1 to 3.

In Formula 1,

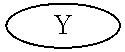

is a compound of a flat aromatic structure, which may be a 5- or 6-membered aryl, a 5- or 6-membered heteroaryl with at least one heteroatom selected from among, N, O, and S, naphthalene, anthracene, triphenylene, pyrene, coronene, triazine, phthalocyanine, porphyrin, or a derivative thereof.

In Formula 1, A may be selected from the group consisting of —(CH₂)ₙ—, —(CH₂)ₙ—S—(CH₂)ₙ—, —(CH₂)ₙ—O—(CH₂)ₙ—, —(CH₂)ₙ—N—(CH₂)ₙ—, —O—C₄H₈—, —O—CH₂—C═CH, —O—CH₂—O—, —O—CO—O—(CH₂)ₙ—, —O—CO—(CH₂)ₙ—, —O—(CH₂)ₙ—NH—, —OCH₂CH₂CH₂SCH₂COO—, —OCH₂CH₂CH₂SCH₂CH₂NH—, and —OC(═O)CH₂CH₂CH₂CH₂CH₂CH₂CH₂CH₂CH₂CH₂CH₂—, wherein n is an integer from 1 to 30.

The flat cyclic compound of Formula 1 may be a compound with C₃-C₂₀ ethenyl or ethynyl group.

An organic compound able to form a polymer capsule by copolymerization with the flat cyclic compound of Formula 1 may be a compound with at least two thiol groups. For example, the organic compound able to form the macromolecule capsule by the copolymerization with the flat cyclic compound of Formula 1 may be a compound represented by Formula 2.

(HS)ⱼ—Z—(SH)ₖ Formula 2

In Formula 2, Z is an unsubstituted or substituted C₁-C₂₀ alkylene, in which O, S, or N may be inserted into the middle of a C—C bond, and the substituent may be selected from the group consisting of HSCH₂CH₂CH₂CH₂CH₂CH₂SH, HSCH₂CH₂OCH₂CH₂OCH₂CH₂SH, HSCH₂CH₂(OH)CH₂(OH)CH₂SH, CH₃CH₂C(CH₂OOCCH₂CH₂SH)₃, and C(CH₂OOCCH₂CH₂SH)₄; and

j and k are each independently an integer from 1 to 3.

The compound of Formula 2 may be a compound selected from the group consisting of 1,8-octanedithiol, 3,6-dioxa-1,8-octanedithiol, pentaerythritol tetrakis(3-mercaptopropionate), trimethylolpropane tri(3-mercaptopropionate), and a combination thereof, but is not limited thereto.

The polymer capsule formed by the copolymerization of the compound of Formula 1 with the compound of Formula 2 may be appropriately formulated and then administered to a patient requiring photodynamic therapy, followed by a photodynamic therapy. The composition for photodynamic therapy according to embodiments of the present invention may be used to treat any disease known to be curable by photodynamic therapy, and in particular, cancers. The types of cancers curable by photodynamic therapy are not specifically limited and may include, for example, liver cancer, lung cancer, uterine cancer, skin cancer, bronchogenic cancer, brain cancer, and gastric cancer.

In the composition for photodynamic therapy including the polymer capsule a separate pharmacologically active agent may be encapsulated as a guest molecule into an internal empty space of the polymer capsule.

The pharmacologically active agent encapsulated into the polymer capsule is not specifically limited and may be any material with pharmacological activity that is soluble or dispersible in a solvent used in the preparation of the polymer capsule. In the composition for photodynamic therapy including the polymer capsule an additional anticancer agent may be encapsulated into the polymer capsule to further enhance an anticancer effect of the photodynamic therapy. Thus, the pharmacologically active agent may be an anticancer agent. Non-limiting examples of the anticancer agent include doxorubicin, daunorubicin, paclitaxel, docetaxel, Taxol, and Glivec. An appropriate anticancer agent may be encapsulated into the polymer capsule, according to the type of cancer to be treated with the composition for photodynamic therapy.

The pharmacologically active agent may be a drug for curing a side effect from the photodynamic therapy. When the composition for photodynamic therapy is used in photodynamic therapy, remarkably less side effects may occur as compared to when using existing photodynamic therapeutic agents. However, a side effect, such as a wound remaining on a body part subjected to the photodynamic therapy, may still occur. Therefore, if a drug with an efficacy to treat or relief such a side effect is encapsulated into the polymer capsule of the composition for photodynamic therapy, at the same time with a photodynamic therapy the side effect of the photodynamic therapy may be relieved or cured. The drug for curing the side effect of the photodynamic therapy may be an anti-inflammatory agent, but is not limited thereto.

The polymer capsule contained in the composition for photodynamic therapy may be prepared according to a method disclosed in KR 721431, the method including: dissolving the compound of Formula 1 and the compound of Formula 2 in an organic solvent; forming the polymer capsule by copolymerizing the compound of Formula 1 and the compound of Formula 2; and removing a compound remaining unreacted without forming the polymer capsule by dialysis.

A method of preparing a polymer capsule into which a pharmacologically active agent is encapsulated may include: dissolving the compound of Formula 1, the compound of Formula 2, and the pharmacologically active agent in an organic solvent; forming the polymer capsule into which pharmacologically active agent is encapsulated, by copolymerizing the compound of Formula 1 and the compound of Formula 2; and removing a compound remaining polymerization or encapsulation by dialysis.

In the method of preparing the polymer capsule, the compound of Formula 1, the compound of Formula 1, and the pharmacologically active agent may be dissolved in the organic solvent irrespective of order. Any one of the compound of Formula 1, the compound of Formula 1, and the pharmacologically active agent may be dissolved first.

The organic solvent that may be used in the above-described methods may be a solvent able to dissolve the compound of Formula 1 and the compound of Formula 2. For example, the solvent may be selected from the group consisting of chloroform, methyl alcohol, ethyl alcohol, dimethyl sulfoxide, dichloromethane, dimethylformamide, tetrahydrofuran, acetone, acetonitrile, and a combination thereof, but is not limited thereto. The amount of the solvent may be a sufficient amount for completely dissolving the compound of Formula 1 and the compound of Formula 2, and the pharmacologically active agent if used.

In the preparation methods described above, the copolymerization of the compound of Formula 1 and the compound of Formula 2 may be performed by a copolymerization method known in the art. For example, UV light may be applied to induce the copolymerization. A UV application for about 6 hours is sufficient to induce and allow most of the reaction to proceed. The UV application duration may be 6 hours or longer. In some embodiments UV of a wavelength of about 256 nm or about 300 nm may be used. The UV may be applied to the reactants at room temperature to induce the copolymerization reaction.

Before the UV application for copolymerization, a radical initiator may be added to the solution of the compound of Formula 1 and the compound of Formula 2 to facilitate the copolymerization reaction of the compound of Formula 1 and the compound of Formula 2. The radical initiator may be selected from the group consisting of AIBN, K₂S₂O₈, (NH₄)₂S₂O₈, benzoyl peroxide, and a combination thereof, but is not limited thereto. Any radical initiator known to one of skill in the art may be used.

The polymer capsule formed according to the above-described method, through the copolymerization of the compound of Formula 1 and the compound of Formula 2, may be identified using scanning electron microscopy (SEM), wherein one droplet of the reaction product solution may be dropped and dried on a planar substrate for observation. The diameter of the polymer capsule may be measured using a dynamic light scattering spectrophotometer.

To prepare the composition for photodynamic therapy with the polymer capsule prepared according to the method described above, the organic solvent in which the polymer capsule is dissolved may be replaced with a physiologically compatible buffer solution by dialysis. Non-limiting examples of the buffer solution include a phosphate-buffered solution (PBS) and 2-[4-(2-hydroxyethyl)-1-piperazinyl]ethanesulfonic acid (HEPES). Any physiologically compatible buffer solution known to one of skill in the art may be used. The solution of the polymer capsule after being replaced with the buffer solution may be formulated as an injection by an injection preparation method widely known in the art.

The composition for photodynamic therapy according to the present invention may be used as a photosensitizer in existing known photodynamic therapy. UV applied in photodynamic therapy may have a wavelength of about 700 nm to about 900 nm. A wavelength of UV that is most absorbable by the polymer capsule may be selected for use. The composition for photodynamic therapy may be administered to the human body by intravenous injection. The dose of the composition for photodynamic therapy may be from about 0.01 mg/kg to about 10 mg/kg. The dose depends on the gender, age, weight, and susceptibility of a patient or type of a disease and may be appropriately controlled according to a doctor's decision.

### Advantageous Effects

As described above, a composition for photodynamic therapy according to the present invention may exhibit remarkably better photosensitization treatment effects with remarkably reduced side effects, as compared to existing photofrin.

### DESCRIPTION OF THE DRAWINGS

FIG. 1 is a graph of survival rates of HeLa cancer cells treated with a phthalocyanine polymer capsule according to an embodiment of the present invention and those not treated with the phthalocyanine polymer capsule, both after photodynamic treatment; and

FIG. 2 is a graph of survival rates of HeLa cancer cells treated with a porphyrin polymer capsule according to an embodiment of the present invention and those not treated with the porphyrin polymer capsule, both after photodynamic treatment.

### BEST MODE

One or more embodiments will now be described in more detail with reference to the following examples. However, these examples are for illustrative purposes only and are not intended to limit the scope of the one or more embodiments.

### MODE OF THE INVENTION

### Example 1

### Preparation of Polymer Capsule Using Octaallyloxyphthalocyanine

1 g (5.01 mmol) of 4,5-dichlorophthalonitrile was dissolved in dimethyl sulfoxide (DMSO), and 1.44 mL (20.3 mmol) of allyl alcohol and 2.81 g (20.3 mmol) of potassium carbonate were added to the solution and stirred at about 50° C. for about 12 hours to obtain a reaction solution. 310 mg (1.25 mmol) of Ni(OAc)₂ was added to the reaction solution and then refluxed for one day. The resulting reaction product was recrystallized using acetonitrile to obtain octaallyloxyphthalocyanine (60 mg, 5%).

¹H NMR (500 MHz, DMSO-d6) δ 11.00 (s, 2H), 6.90 (s, 8H), 6.80 (s, 4H), 5.89 (d, 8H), 5.24 (dd, 8H), 5.20 (dd, 8H), 4.61 (s, 16H); MS (FAB) m/z 958.41 [M]⁺.

After 9.6 mg of the octaallyloxyphthalocyanine was completely dissolved in about 10 mL of toluene, 40 mg of 1,3-dioxa-2,8-octanedithiol was added to the solution and dissolved. UV light having wavelengths of about 256 nm and about 300 nm was applied for about 6 hours, followed by dialysis. Using a PBS buffer solution as a dialysis solution, residues of octaallyloxyphthalocyanine and 1,3-dioxa-2,8-octanedithiol remaining without polymerization were removed by dialysis.

### Example 2

### Photodynamic Therapy that Uses Octaallyloxyphthalocyanine Polymer Capsule

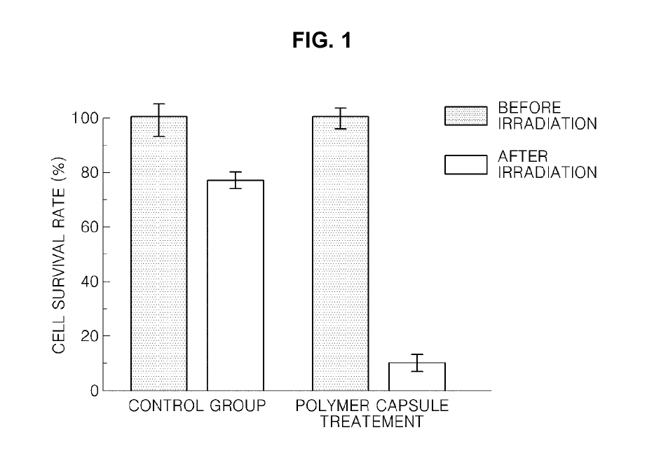

After 180 μl of a Dulbecco's modified eagle's medium (DMEM) was put into a plastic container in which about 5,000 HeLa cells had been cultured, a 0.135 mg/mL dispersion of the octaallyloxyphthalocyanine polymer capsule prepared in Example 1 in 20 μl of a PBS buffer solution (pH 7.2) was added into the plastic container and cultured in an incubator containing about 5% CO₂ at about 37° C. One hour later, the cultured product was irradiated by an infrared (IR) lamp emitting light with a wavelength of 700 nm for about 12 hours in a dark room. After the cultured product was cultured one day further with the IR lamp turned off, an MTT assay was conducted to measure a cell survival rate of HeLa cells treated with the polymer capsule and those not treated with the polymer capsule. The observation results from the MTT assay are shown in FIG. 1.

As shown in FIG. 1, the cells treated with the polymer capsule were found to have a survival rate of about 10% of that of those not treated with the polymer capsule, confirming that HeLa cancer cells may be photodynamically killed using the phthalocyanine polymer capsule.

### Example 3

### Preparation of Polymer Capsule Using tetra(3,5-bisallyloxyphenyl)porphyrin

2 g (9.15 mmol) of 3,5-bisallyloxybenzaldehyde and 615 mg (9.15 mmol) of pyrrole were dissolved in chloroform, and 40 μl of a trifluoroboron/diethyl ether complex was added thereto in a nitrogen gas atmosphere and stirred for about one day. After removing the solvent, the residue was isolated by column chromatography using hexane and chloroform, followed by recrystallization in chloroform and methanol to obtain 150 mg of tetra(3,5-bisallyloxyphenyl)porphyrin with a yield of 6%.

¹H NMR (500 MHz, CDCl3) δ 11.40 (s, 2H), 6.42 (s, 8H), 6.33 (s, 4H), 6.10 (s, 8H), 5.89 (d, 8H), 5.24 (dd, 8H), 5.20 (dd, 8H), 4.61 (s, 16H); MS (FAB) m/z 1062.29 [M]⁺.

10.6 mg of the tetra(3,5-bisallyloxyphenyl)porphyrin was completely dissolved in about 10 mL of toluene, and then 40 mg of 1,3-dioxa-2,8-octanedithiol was added thereto and dissolved. After a UV application with a wavelength of about 256 nm and 300 nm for about 6 hours, dialysis was performed using a PBS buffer solution as a dialysis solution to remove tetra(3,5-bisallyloxyphenyl)porphyrin and 1,3-dioxa-2,8-octanedithiol remaining without polymerization.

### Example 4

### Photodynamic Therapy that Uses tetra(3,5-bisallyloxyphenyl)porphyrin Polymer Capsule

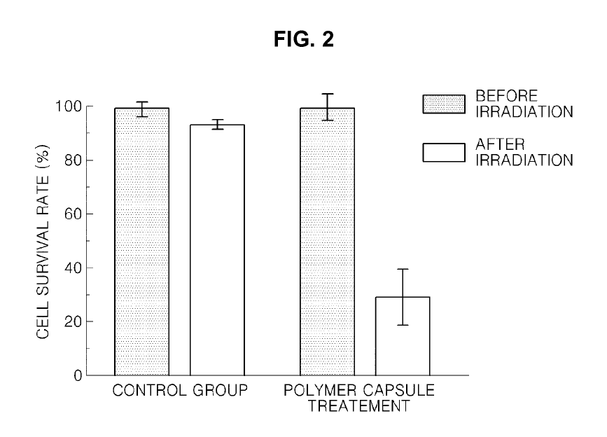

After 180 μl of a DMEM was put into a plastic container in which about 5,000 HeLa cells had been cultured, a 0.135 mg/mL dispersion of the tetra(3,5-bisallyloxyphenyl)porphyrin polymer capsule prepared in Example 3 in 20 μl of a PBS buffer solution (pH 7.2) was added into the plastic container and cultured in an incubator containing about 5% CO₂ at about 37° C. One hour later, the cultured product was irradiated by an IR lamp emitting light with a wavelength of 630 nm for about 12 hours in a dark room. After the cultured product was cultured one day further with the IR lamp turned off, an MTT assay was conducted to measure a cell survival rate of HeLa cells treated with the polymer capsule and those not treated with the polymer capsule. The observation results from the MIT assay are shown in FIG. 2. As shown in FIG. 2, the cells treated with the polymer capsule were found to have a survival rate of about 30% of that of those not treated with the polymer capsule. This confirms that HeLa cancer cells may be killed by photodynamic therapy using the porphyrin polymer capsule.

### Example 5

### Photodynamic Therapy that Uses Polymer Capsule on Animal Model

About 20 g-weight mice transplanted with breast cancer cell tissues of about 6-10 mm in size were prepared, and a 0.135 mg/mL dispersion of the octaallyloxyphthalocyanine polymer capsule (Example 1) in pH 7.2 phosphate buffer solution, 0.135 mg/mL dispersion of the tetra(3,5-bisallyloxyphenyl)porphyrin polymer capsule (Example 3) in a pH 7.2 phosphate buffer solution, or 1 mg/mL of photofrin (Axcan Pharma Inc., U.S.A) was injected in an amount of about 200 μl into tail blood vessels of the mice. The mice were then irradiated by an IR lamp emitting light with a wavelength of about 630-700 nm for about 72 hours in a dark room. After the IR lamp was turned off, treatment effects in the mice medicated with the polymer capsule, photofrin II, or nothing were observed. The treatment effects in each group were investigated with respect to the number of cancer cell-killed mice, tissue damage score (TDS), and functional damage score (FDS). TDS means a degree of tissue damage in cancer treatment sites, and FDS means a degree of normal functioning in the cancer treatment sites.

The results of the animal test are shown in Table 1 below.

TABLE 1

| | Number of cancer | Average | Average |
|---|---|---|---|
| | cell-killed mice per | TDS after | FDS after |
| | tens of subjects | 44 days | 44 days |
| Phthalocyanine | 8 | 1.25 | 1.25 |
| polymer capsule | | | |
| Porphyrin polymer | 7 | 1.13 | 1.38 |
| capsule | | | |
| Photofrin II | 7 | 1.29 | 4.0 |

As shown in Table 1 above, about 80% of the mice treated with the phthalocyanine polymer capsule were cured from cancer, and about 70% of those treated with the polymer capsule recovered from cancer. Similar to the group treated with the polymer capsule, about 70% of the mice were cured from cancer when treated with photofrin II, an FDA-approved photodynamic therapeutic agent. Whether the legs with cancer tissues recovered to normally function after the photodynamic therapy was investigated. As a result, the group treated with the polymer capsule could normally function despite wounds still remaining. However, the group treated with photofrin II was abnormal in leg function even after the photodynamic therapy. The groups treated with the phthalocyanine or porphyrin polymer capsule exhibit a similar result to the group treated with the photofrin in a concentration of one eighth to one seventh of the concentration of the photofrin. Therefore, the polymer capsule according to embodiments of the present invention is found to have remarkably better effect in photodynamic therapy and less side effects as compared to when existing photodynamic therapeutic agents are used.

While the inventive concept has been particularly shown and described with reference to exemplary embodiments thereof, it will be understood that various changes in form and details may be made therein without departing from the spirit and scope of the following claims.

1. A composition for photodynamic therapy comprising a polymer capsule having a diameter of about 10 nm to about 2000 nm synthesized by copolymerization of a compound represented by Formula 1 below and a compound represented by Formula 2 below: 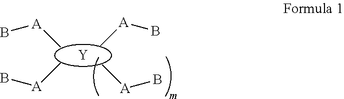
wherein, in Formula 1, 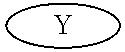
is a C₅-C₅₄ aryl group, or a C₅-C₅₄ heteroaryl group with at least one heteroatom selected from among N, O, and S; A is independently selected from the group consisting of —(CH₂)ₙ—, —(CH₂)ₙ—S—(CH₂)ₙ—, —(CH₂)ₙ—O—(CH₂)ₙ—, —(CH₂)ₙ—N—(CH₂)ₙ—, —O—, —O—(CH₂)ₙ—, —O—(CH₂)ₙ—C═C—, —O—(CH₂)ₙ—O—, —O—CO—(CH₂)ₙ—, —O—CO—O—(CH₂)ₙ—, —O—(CH₂)ₙ—NH—, —O—(CH₂)ₙ—S—(CH₂)ₙ—COO—, and —(CH₂)ₙ—S—(CH₂)ₙ—NH—, wherein n is an integer from 0 to 30; B is —CH═CH₂ or —C≡CH; and m is an integer from 0 to 20,
(HS)ⱼ—Z—(SH)ₖ Formula 2
wherein, in Formula 2, Z is an unsubstituted or substituted C₁-C₂₀ alkylene, in which O, S, or N may be inserted into the middle of a C—C bond, and the substituent is selected from the group consisting of —SCH₂CH₂CH₂CH₂CH₂CH₂SH, —SCH₂CH₂OCH₂CH₂OCH₂CH₂SH, —SCH₂CH₂(OH)CH₂(OH)CH₂SH, —CH₂CH₂C(CH₂OOCCH₂CH₂SH)₃, and —C(CH₂OOCCH₂CH₂SH)₃; and j and k are each independently an integer from 1 to 3.

2. The composition of claim 1, wherein 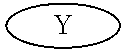
is a 5- or 6-membered aryl, a 5- or 6-membered heteroaryl with at least one heteroatom selected from among N, O, and S, naphthalene, anthracene, triphenylene, pyrene, coronene, triazine, phthalocyanine, porphyrin, or a derivative thereof; and A is selected from the group consisting of —(CH₂)—, —(CH₂)ₙ—S—(CH₂)ₙ—, —(CH₂)ₙ—O—(CH₂)ₙ—, —(CH₂)ₙ—NH—(CH₂)ₙ—, —O—, —O—CH₂—, —O—C₄H₈—, —O—CH₂—C═C—, —O—CH₂—O—, —O—CO—O—(CH₂)ₙ—, —O—CO—(CH₂)ₙ—, —O—(CH₂)ₙ—NH—, —OCH₂CH₂CH₂SCH₂COO—, —OCH₂CH₂CH₂SCH₂CH₂NH—, and —OC(═O)CH₂CH₂CH₂CH₂CH₂CH₂CH₂CH₂CH₂CH₂CH₂—, wherein n is an integer from 1 to 30.

3. The composition of claim 1, wherein the compound of Formula 1 has a C₃-C₂₀ ethenyl (—CH═CH₂) or ethynyl (—C≡CH) group.

4. The composition of claim 1, wherein the compound of Formula 2 is selected from the group consisting of 1,8-octanedithiol, 3,6-dioxa-1,8-octanedithiol, pentaerythritol tetrakis(3-mercaptopropionate), trimethylolpropane tri(3-mercaptopropionate), and a combination thereof.

5. The composition of claim 1, wherein the composition is for the treatment of liver cancer, lung cancer, uterine cancer, skin cancer, bronchogenic cancer, brain cancer, or gastric cancer.

6. The composition of claim 1, wherein a pharmacologically active agent as a guest material is encapsulated into an internal part of the polymer capsule.

7. The composition of claim 6, wherein the pharmacologically active agent is an anti-cancer agent.

8. The composition of claim 7, wherein the anti-cancer agent is selected from the group consisting of doxorubicin, daunorubicin, paclitaxel, docetaxel, Taxol, and Glivec.

9. The composition of claim 6, wherein the pharmacologically active agent is a drug for the treatment of a side effect of photodynamic therapy.
